(19) United States
(12) Patent Application Publication
(10) Pub. No.: US 2025/0099446 A1
(43) Pub. Date: Mar. 27, 2025

(54) HYBRID PHARMACEUTICAL COMPOSITION OBTAINED BY CONJUGATION OF A PROTON PUMP INHIBITOR AND A CARBON ANHYDRASE INHIBITOR

(71) Applicant: EXO LAB ITALIA, S.r.l., Pescara (IT)

(72) Inventors: Stefano FAIS, Pescara (IT); Mariantonia LOGOZZI, Pescara (IT); Alessio NOCENTINI, Pescara (IT); Claudiu Trandafir SUPURAN, Pescara (IT); Silvia BUA, Pescara (CN)

(73) Assignee: EXO LAB ITALIA, S.r.l., Pescara (IT)

(21) Appl. No.: 18/729,118

(22) PCT Filed: Jan. 14, 2023
(86) PCT No.: PCT/IB2023/050347

§ 371 Date: Jul. 15, 2024

(30) Foreign Application Priority Data
Jan. 21, 2022 (IT) 102022000001022

(51) Int. Cl. A61K 31/4439; A61P 7/08; C07D 419/14

(52) U.S. Cl. CPC A61K 31/4439; A61P 7/08; C07D 419/14

(57) ABSTRACT

A pharmaceutical compositions (A, G1-G8) capable of inhibiting the ATP-dependent proton pump (V-ATPase) and carbonic α-anhydrases (CA IV, IX and XII), having the general formulae: Formula A (PPI-CAI), G1, G2, G3, G4, G5, G6, G7 and G8 and a pharmaceutically acceptable excipient.

### BACKGROUND OF THE INVENTION

### 1. Field of the Invention

The present invention relates to new hybrid pharmaceutical composition obtained by conjugating proton pump inhibitors (PPI) with carbonic anhydrase inhibitors (CA) and their salts, as well as new formulations thereof. The invention also proposes new uses for the treatment of diseases characterized by loco acidosis based on the activation of hybrid pro-drugs in the target tissue. The invention also proposes new uses of these compositions to reduce the acidity in the target tissue by means of a multi-target strategy consisting in the inhibition of proton pumps concomitant to the inhibition of carbonic anhydrases. The compositions are advantageously used to treat disorders having acidotic characteristics such as, but not limited to, hypoxic tumors, gastrointestinal disorders, inflammation, arthritis, pathogenic infections; that is, it aims to decrease or reduce the gastrointestinal toxicity associated with the use of nonsteroidal anti-inflammatory compounds.

### 2. Brief Description of the Prior Art

As is known, pH regulation in all tissues and organisms is a tightly controlled process involving a multitude of biochemical mechanisms. For example, the growth of solid tumors is characterized by pH imbalances both inside and outside the cell, also related to changes in the tumor environment that support the growth of the tumor mass and the development of metastases (Hanahan D, Weinberg R A Hallmarks of cancer: the next generation Cell 2011, 144, 646-674). The extra oxygen and nutrients required by the developing tumor are supplied by the formation of new, often dysfunctional blood vessels from the pre-existing ones (angiogenesis) (Neri D, Supuran C T. Interfering with pH regulation in tumors as a therapeutic strategy. Nat Rev Drug Discov. 2011, 10,767-77). The high tumor metabolic rate therefore often leads to acidosis and hypoxia due to poor perfusion. Indeed, an upregulated glucose metabolism is a hallmark of hypoxic and highly invasive tumors, due to inadequate oxygen supply limiting oxidative phosphorylation. As a result, hypoxic cancer cells shift their metabolism towards glycolysis, a less energy-efficient but oxygen-independent process. Glycolysis often persists even after reoxygenation because the metabolic intermediates produced (lactate and pyruvate) can be utilized for the biosynthesis of amino acids, nucleotides and lipids, thus providing a selective advantage to tumor cell proliferation. The high glucose consumption and high lactate production in tumor tissues is known as the Warburg effect (Fang J S, Gillies R J, Gatenby, R A. Adaptation to hypoxia and acidosis in carcinogenesis and tumor progression. Sem. Cancer Biol. 2008, 18, 330-337). The metabolic shift therefore produces an excess of protons and carbon dioxide, which are not compatible with basic cellular functions. Therefore, cancer cells to cope with this acid and hypoxic stress have developed various mechanisms that activate ion exchangers, pumps and transporters (FIG. 1) such as sodium-proton exchangers (NHE1), anion exchangers (AE2), sodium bicarbonate transporters (NBCel), V-ATPases, monocarboxylate transporters (MCT4), glucose transporters (GLLIT1) and carbonic anhydrases (CA), which maintain a slightly alkaline internal pH (pHi) by acidifying the extracellular environment (pHe in the range 6, 5-7.0). Hypoxia also constitutes an impediment for radiotherapy, as oxygen is required to oxidize radiation-induced DNA free radicals which subsequently lead to cancer cell death. Furthermore, the role of carbonic anhydrases associated with tumors is further known. a-Carbonic anhydrases (CA, EC 4.2.1.1) are metalloenzymes widespread in higher vertebrates, including humans, which act as catalysts for the reversible hydration of carbon dioxide to bicarbonate and protons (CO2+H2O<−HCO3−+H+), a vital reaction for all organisms on Earth (Supuran, C. T. Carbonic anhydrases: novel therapeutic applications for inhibitors and activators. Nature Rev. Drug Discov. 2008, 7, 168-181). Fifteen isoforms are present in humans, and they differ in subcellular localization, catalytic activity, and susceptibility to different classes of inhibitors. The isoforms CA IX and CA XII, bound externally to the cell membrane, are overexpressed in many cancers and are associated with cancer progression and metastasis. CA IX is normally expressed in the stomach and peritoneal lining, but is strongly overexpressed in several types of solid tumors, mainly by the action of hypoxia-inducible transcription factor 1 (HIF1), as described by Nocentini A, Supuran C T. Carbonic anhydrase inhibitors as antitumor/antimetastatic agents: a patent review (2008-2018), Expert Opin Ther Pat. 2018, 28, 729-740. The gene expressing CA IX is the one most strongly overexpressed in response to hypoxia in human cancer cells. CA XII, normally more prevalent in healthy tissue, is also overexpressed in many cancers. CA IX and XII have represented the main target of scientific research in the field of CA in the last two decades until their validation as drug targets for anticancer therapies. In fact, the inhibition of these two isoforms of CA with inhibitors (CAI) slows down the growth, invasiveness and metastasis of the primary tumor and reduces the increase in the population of tumor stem cells. A multitude of multidisciplinary studies led in 2016 to the entry into clinical phase I of the first CA inhibitor, SLC-0111 a primary sulfonamide currently in phase Ib/II, for the treatment of hypoxic tumors (ClinicalTrials.gov, https://clinicaltrials. gov/ct2/results?term=slc0111&Search=Search—Accessed on May 14, 2018). It is also known by Perez-Sayans M, Somoza-Martin J M, Barros-Angueira F, Rey J M, Garcia-Garcia A. V-ATPase inhibitors and implication in cancer treatment. Cancer Treat. Rev. 2009, 35, 707-713 and by Spugnini E, Citro G, Fais S. Proton pump inhibitors as anti vacuolar-ATPases drugs: a novel anticancer strategy. J. Exp. Clin. Cancer Res. 2010, 29, 44, that V-ATPase is an ATP-dependent proton pump involved in the acidification of intracellular compartments and in the extrusion of protons across the cytoplasmic membrane. It consists of multiple subunits with cytosolic and transmembrane domains. The similarity between V-ATPase and H+-K+ ATPase, involved in stomach acid secretion, has aroused interest in studying proton pump inhibitors (PPIs) for their ability to inhibit V-ATPase as shown in FIG. 1. These compositions, including omeprazole, pantoprazole, lansoprazole and rabeprazole, are widely used clinically as antacids and are prodrugs that require activation in an acidic environment where they undergo a chemical transformation producing a sulfenamide capable of reacting with cysteine residues of the proton pump inactivating it. V-ATPase inhibition has been shown to trigger rapid tumor cell death due to intracellular acidification, caspase activation, and accumulation of reactive oxygen species, as per Fais, S. Proton pump inhibitor-induced tumor cell death by inhibition of a detoxification mechanism. J. Intern. Med. 2010, 267, 515-525. Many human tumors, including melanomas, osteosarcomas, lymphomas and various adenocarcinomas, respond to PPIs, as by Spugnini E, Citro G, Fais S. Proton pump inhibitors as anti vacuolar-ATPases drugs: a novel anticancer strategy. J. Exp. Clin. Cancer Res. 2010, 29, 44. Metastatic tumors are even more sensitive to PPIs as they are generally more acidic than most primary tumors. Lansoprazole has been shown to be the most effective and promising agent against cancer cells within the PPI class. Further studies, Azzarito, T.; Venturi, G.; Cesolini, A.; Fais, S. Lansoprazole induces sensitivity to suboptimal doses of paclitaxel in human melanoma, Cancer Lett. 2015, 356, 697-703 and Luciani, F.; Sword, M.; DeMilito, A.; Molinari, A.; Rivoltini, L.; Montinaro, A.; Marra, M.; Lugini, L.; Logozzi, M.; Lozupone, F.; et al. Effect of proton pump inhibitor pretreatment on resistance of solid tumorsto cytotoxic drugs. J. Natl. Cancer Inst. 2004, 96, 1702-1713, demonstrated that the antitumor activity of PPIs can be synergistic with classical anticancer agents or with molecules active towards completely different cellular systems such as reverse transcriptase (Lessi E, Logozzi M, Mizzoni D, Di Raimo R, Supuran C T, Fais S. Rethinking the Combination of Proton Exchanger Inhibitors in Cancer Therapy. Metabolites. 2017 Dec. 23; 8 (1), pii: E2. doi: 10.3390/metabo801000) or CA IX (Lugini L, Sciamanna I, Federici C, Iessi E, Spugnini E P, Fais S. Antitumor effect of combination of the inhibitors of two new oncotargets: proton pumps and reverse transcriptase, Oncotarget. 2017 Jan. 17; 8 (3): 4147-4155). Thanks to the scientific background of the inventors and the leadership in this research field, the present invention proposes an innovative class of hybrid compositions combining a PPI and an inhibitor of CA IX and XII for the treatment of diseases characterized by loco acidosis such as hypoxic tumors and metastatic (Logozzi M, Mizzoni D, Angelini D F, Di Raimo R, Falchi M, Battistini L, Fais S. Microenvironmental pH and Exosome Levels Interplay in Human Cancer Cell Lines of Different Histotypes. Cancers (Basel). 2018 Oct. 5; 10 (10): 370. doi: 10.3390/cancers 10100370).

Thus, the involvement of the ATP-dependent proton pump (V-ATPase) and a-carbonic anhydrases (CA) in the pH modulation mechanism of hypoxic tumors has guided the design of novel molecular hybrids incorporating scaffolds able to interfere with both the targets. In recent years, the multi-target pharmaceutical approach has been attracting growing interest with respect to the co-administration of multiple drugs, given the various therapeutic benefits it can bring as a function of the improved pharmacokinetic and pharmacodynamic properties of the hybrid.

There is therefore the need to identify new pharmaceutical compositions for the purposes set out above.

### SUMMARY OF THE INVENTION

The object of the present invention is therefore new pharmaceutical compositions comprising combinations of proton pump inhibitors (PPIs) active against the ATP-dependent proton pump (V-ATPase) with chemotypes (sulfonamides, coumarins, sulfocoumarin, benzoxaboroles, mono- and dithiocarbamates, etc.) capable of producing effective inhibition of tumor a-carbonic anhydrase (CA).

According to the present invention, molecular hybrids with pro-drug characteristics have been obtained which possess unique pharmacokinetics and release the two active ingredients under suitable conditions, i.e. those that distinguish hypoxic tumors, second example shown in FIG. 2. Then, PPIs were functionalized via imidazole NH using a carbamate, monothiocarbamate or dithiocarbamate linker which can undergo rapid hydrolysis in the acidic conditions present in the extracellular microenvironment of tumors. Advantageously, following the hydrolysis of the carbamate prodrug, the released PPI can undergo, as a prodrug in turn, the activation that allows it to interact and block the V-ATPase. At the same time, both the full hybrid and the CAI released following hydrolysis of the carbamate linker can interfere with the activity of target CAs by blocking, together with V-ATPase inhibition, extracellular acidification. Thus, according to the present invention, new pharmaceutical compositions are defined, as specified in the attached independent product claim.

According to a further aspect of the present invention, new uses of such pharmaceutical compositions are defined, as specified in the attached independent claims of use. The dependent claims outline particular and further advantageous aspects of the invention.

### BRIEF DESCRIPTION OF THE DRAWINGS

These and other advantages of the invention will now be described in detail, with reference to the accompanying drawings, which represent an exemplary embodiment of the invention, wherein:

FIG. 1 shows the molecular structure of proton pump inhibitors used as antacids;

FIG. 2 shows an example of activation of the proposed pro-drugs with release of the single active components, depicted with a representative hybrid of lansoprazole with coumarin (CAI);

FIG. 3 shows the general structure A of the hybrids proposed in this invention, i.e. PPI connected via imidazole NH and carbamate, monothiocarbamate or dithiocarbamate linkers to G1-G8 structures of CAI chemotypes;

FIG. 4 shows an example of a synthetic strategy adopted for the synthesis of PPI/CAI hybrid prodrugs;

FIG. 5 shows examples of synthetic strategies adopted to include alcohol or thiol groups on CAI scaffolds;

FIG. 6 shows the results of cell mortality at pH 7.4 on melanoma;

FIG. 7 shows the results of cell mortality at pH 6.5 on melanoma;

FIG. 8 shows the results of cell mortality at pH 7.4 on glioblastoma;

FIG. 9 shows the results of cell mortality at pH 6.5 on glioblastoma;

FIG. 10 the results of cell mortality at pH 6.5 for prostate cancer;

FIG. 11 shows the results of cell mortality at pH 6.5 on peripheral blood mononuclear cells cultured at pH 7.4.

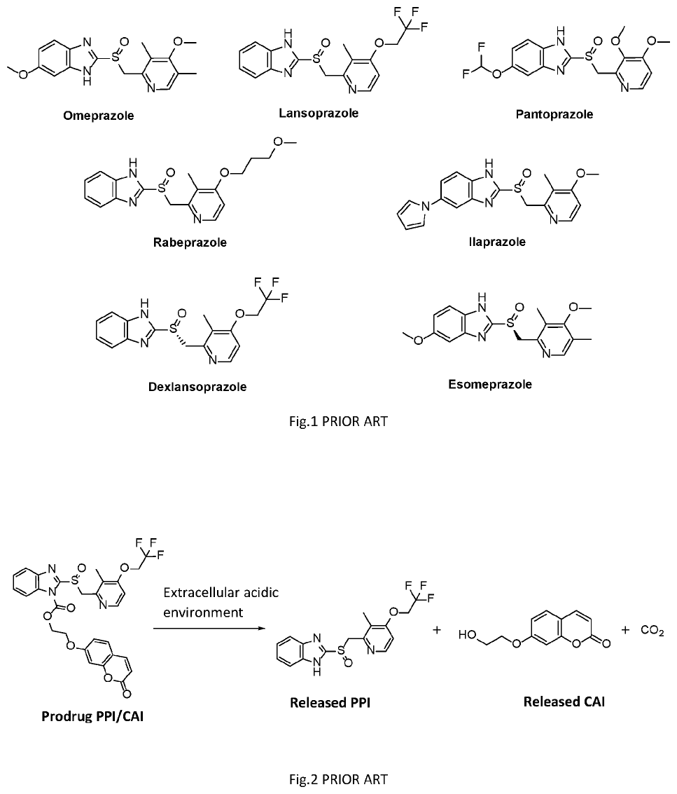

### DETAILED DESCRIPTION OF THE PREFERRED EMBODIMENTS

According to the present invention as shown in FIG. 2, the activation of the pro-drug with release of the individual active components is depicted with a representative hybrid lansoprazole with coumarin (CAI). The double priming in an acid environment of the pro-drug allows a further enhancement of the specificity of action of these agents against hypoxic and acid tumors, compared to healthy tissues.

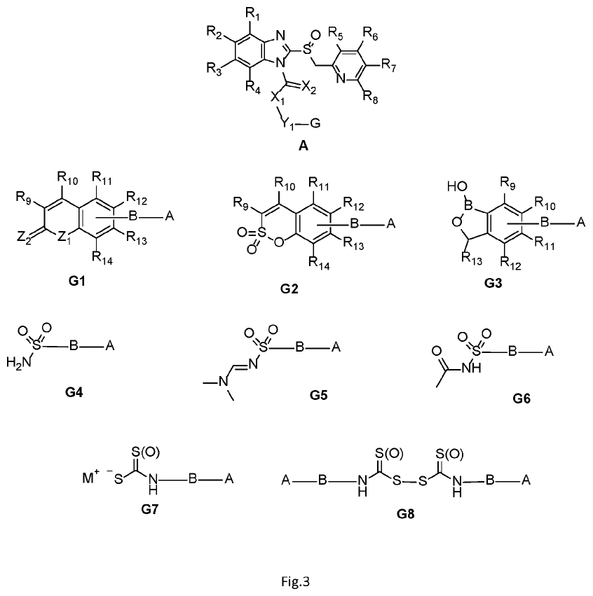

On the basis of the aforementioned design, various CAI G1-G8 chemotypes (primary aromatic and aliphatic sulfonamides, coumarins, sulfocoumarins, benzoxaborols, mono/dithiocarbamates, etc.) were equipped with aliphatic alcoholic or thiol moieties prepared for hybridization with PPI forming a linker of carbamate nature, as shown in FIG. 3. Spacers of variable nature and length to connect the two active components are proposed in order to evaluate the combinations that allow the best release of the components and the inhibition of the CAs.

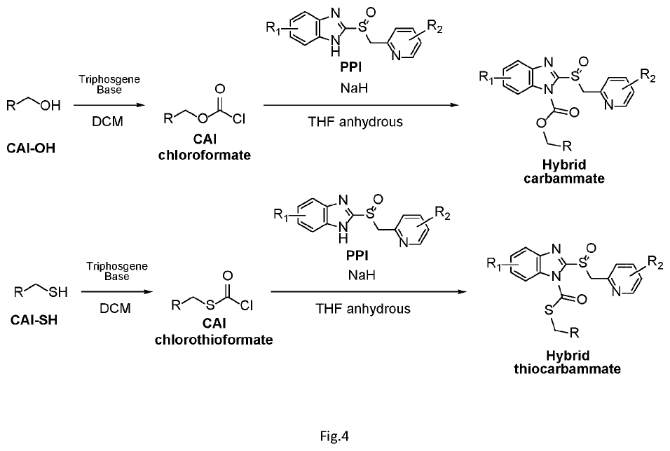

As shown in FIG. 4, the main synthesis procedure of the hybrid prodrugs involves the conversion of the alcohol or thiol function present on the CAI scaffold (CAI-OH or CAI-SH) into the corresponding chloroformate or chlorothioformate using appropriate agents, such as triphosgene or thiophosgene. Said isolated intermediates are reacted with the PPI in the form of sodium salt obtained by treating the PPI with NaH in suitable solvents.

According to the present invention, various synthetic procedures have been adopted to functionalize the CAI scaffolds (primary aromatic and aliphatic sulfonamides, coumarins, sulfocoumarins, benzoxaborols, mono/dithiocarbamates, etc.) with alcoholic or thiol functions, necessary for the reaction described above: for example substitutions nucleophiles, coupling reactions, Click Chemistry, etc. Further derivatizations were carried out on the sulfonamide function (e.g. protection with acetyl or DMF dialkylacetal) or thiocarbamate (e.g. S—S dimerization).

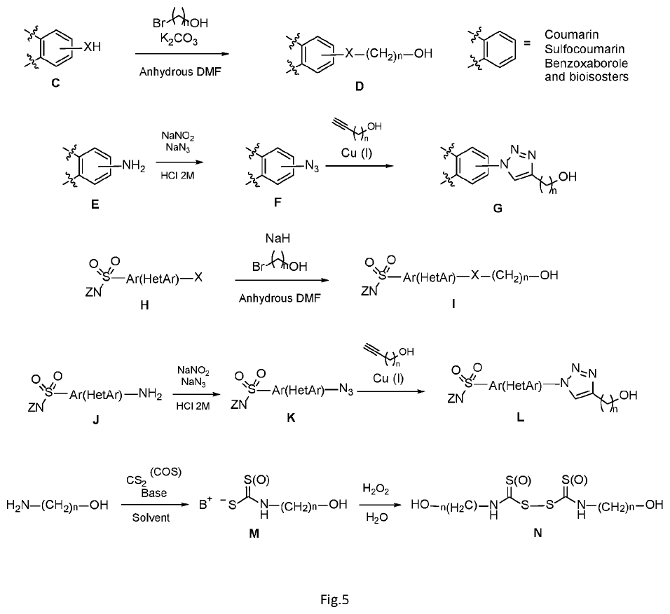

Examples of synthetic strategies adopted to include alcohol or thiol groups on CAI scaffolds are shown in FIG. 5. All compounds were purified by silica gel chromatography and characterized by 1H, 13C, 19F NMR, LC-MS, etc.

In particular, a general procedure for the synthesis of the series of hybrid derivatives A1-A4 and their characterization is described below. To a solution of a specific alcohol G1A-G1 D (0.3 g, 1.0 eq.) and triethylamine (1.0 eq.) in anhydrous tetrahydrofuran (4 mL) at 0° C. in an inert nitrogen atmosphere, triphosgene (0.95 eq.) is added. The reaction mixture is stirred at rt. up to the consumption of the starting products (monitoring via TCL). The formed precipitate is removed by filtration and the filtrate is concentrated in vacuo. The corresponding chloroformates thus obtained are then dissolved in anhydrous tetrahydrofuran (4 mL) and this mixture is added dropwise to a solution of Lansoprazole (1.0 eq.) and NaH (1.0 eq) in anhydrous tetrahydrofuran (4 mL). The reaction mixture is mixed at t.a. until consumption of the starting products (monitoring by TCL), treated with ice and extracted with ethyl acetate (3×10 mL). The combined organic phase is washed with a saturated solution of NaCl, dried with Na2SO4, filtered and concentrated in vacuo to give the crude derivative. The latter is purified by column chromatography with silica gel as the stationary phase and a mixture of 1% methanol in dichloromethane as the mobile phase to obtain compounds A1-A4. A1: 2-((2-Osso-2H-cromen-6-il)ossi)etil 2-(((3-metil-4-(2,2,2-trifluoroetossi)piridin-2-il)metil)sulfinil)-1H-benzo[d]imidazolo-1 carbossilato.

Compound A1 was obtained as a white powder according to the above general procedure using the alcohol 6-(2-hydroxyethoxy)-2H-chromen-2-one G1A. Yield 14%; mp 163-165° C.; TLC: Rf=0.43 (methanol/dichloromethane 10% v/v); 1H-NMR (DMSO-d6, 400 MHZ): 5 2.16 (3H, s, CH3), 4.53 (3H, m, SO—CH2, CH2-CH2-O), 4.88 (5H, m, CO—CH2-CH2, CH2-CH2-O, CH2-CF3), 6.52 (1H, d, J=9.6, Ar—H), 7.01 (1H, d, J=5.6, Ar—H), 7.24 (1H, dd, J=8.6, 3.6, Ar—H), 7.34 (2H, m, pyridine-CH, Ar—H), 7.50 (2H, m, Ar—H), 7.85 (1H, d, J=6.8, Ar—H), 8.0 (1H, d, J=9.6, Ar—H), 8.05 (1H, d, J=7.6, Ar—H), 8.17 (1H, d, J=5.6, pyridine-CH); 13C-NMR (DMSO-d6, 100 MHz): 5 11.4, 60.2, 65.5 (J2C—F=34), 67.2, 67.8, 107.6, 112.7, 115.7, 117.6, 118.3, 120.1, 120.6, 121.4, 122.9, 124.7 (J1C—F=276), 125.8, 127.1, 134.4, 143.0, 144.8, 148.6, 149.0, 149.9, 152.5, 155.2, 159.1, 160.9, 162.0; 19F-NMR (DMSO-d6, 376 MHZ): 5-72.7 (3F, s); MS (ESI positive) m/z=602.1 [M+H]+.

A2: 2-((2-Oxo-2H-chromen-7-yl)oxy)ethyl 2-(((3-methyl-4-(2,2,2-trifluoroethoxy)pyridin-2-yl)methyl)sulfinyl)-1H-benzo[d]imidazole-1 carboxylate.

Compound A2 was obtained as a white powder according to the above general procedure using alcohol 7-(2-idrossietossi)-2H-cromen-2-one G1 B. Resa 61.0%; pf 201-203° C.; TLC: Rf=0.13 (methanol/dichloromethane 10% v/v); 1H-NMR (DMSO-d6, 400 MHZ): 5 2.12 (3H, s, CH3), 4.49 (1H, d, J=14.0, CH2-CH2-O), 4.55 (2H, t, J=8.8, 4.4, SO—CH2), 4.84 (5H, m, CO—CH2-CH2, CH2-CH2-O, CH2-CF3), 6.30 (1H, d, J=9.6, Ar—H), 6.94 (1H, dd, J=8.6, 3.6, Ar—H), 7.98 (1H, d, J=5.8, Ar—H), 7.01 (1H, d, J=2.4, Ar—H), 7.47 (2H, m, Ar—H), 7.60 (1H, d, J=8.6, pyridine-CH), 7.82 (1H, m, Ar—H), 7.97 (1H, d, J=9.6, Ar—H), 8.01 (1H, m, Ar—H), 8.13 (1H, d, J=5.6, pyridine-CH); 13C-NMR (DMSO-d6, 100 MHZ): 13C-NMR (DMSO-d6, 100 MHz): 5 11.4, 60.2, 65.5 (J2C—F=34), 66.8, 67.6, 102.3, 107.6, 113.5, 113.6, 113.7, 115.7, 121.4, 122.9, 124.7 (J1C—F=276), 125.9, 127.1, 130.4, 134.4, 143.0, 145.1, 148.5, 149.8, 152.5, 156.1, 159.1, 161.1, 161.9, 162.0; 19F-NMR (DMSO-d6, 376 MHZ): 5-72.7 (3F, s); MS (ESI positive) m/z=602.1 [M+H]+.

A3: 3-((2-Oxo-2H-chromen-6-yl)oxy)propyl 2-(((3-methyl-4-(2,2,2-trifluoroethoxy)pyridin-2-yl)methyl)sulfinyl)-1H-benzo[d]imidazole-1-carboxylate. Compound A3 was obtained as a white powder according to the above general procedure using 6-(3-hydroxypropoxy)-2H-chromen-2-one G1C alcohol. Yield 15%; mp 123-125° C.; TLC: Rf=0.48 (methanol/dichloromethane 10% v/v); 1H-NMR (DMSO-d6, 400 MHZ): 52.19 (3H, s, CH3), 2.36 (2H, m, CH2-CH2-CH2), 4.24 (2H, t, J=12, 6, SO—CH2), 4.52 (1H, d, J=14, CO—CH2-CH2), 4.70 (2H, m, CH2-CH2-O), 4.91 (3H, m, CH2-CF3, CO—CH2-CH2), 6.49 (1H, d, J=9.6, Ar—H), 7.06 (1H, d, J=5.6, Ar—H), 7.18 (1H, dd, J=8.6, 3.6, Ar—H), 7.27 (1H, d, J=2.8, Ar—H), 7.32 (1H, d, J=8.8, pyridine-CH), 7.66 (2H, m, Ar—H), 7.86 (1H, d, J=7.2, Ar—H), 7.96 (1H, d, J=9.6, Ar—H), 8.05 (1H, d, J=7.6, Ar—H), 8.20 (1H, d, J=5.6, pyridine-CH); 13C-NMR (DMSO-d6, 100 MHz): 5 11.5, 28.6, 60.3, 65.3, 65.8 (J2C—F=9), 66.9, 107.7, 112.3, 115.7, 117.5, 118.2, 120.0, 120.6, 121.4, 123.0, 124.7 (J1C—F=276), 125.8, 127.1, 134.4, 143.0, 144.8, 148.6, 148.8, 150.1, 152.6, 155.6, 159.0, 161.0, 162.1; 19F-NMR (DMSO-d6, 376 MHZ): 5-72.7 (3F, s); MS (ESI positive) m/z=616.1 [M+H]+.

A4: 3-((2-Oxo-2H-chromen-7-yl)oxy)propyl 2-(((3-methyl-4-(2,2,2-trifluoroethoxy)pyridin-2-yl)methyl)sulfinyl)-1H-benzo[d]imidazole-1-carboxylate. Compound A4 was obtained as a white powder according to the above general procedure using the alcohol 7-(3-hydroxypropoxy)-2H-chromen-2-one G1 D. Yield 26%; mp 108-110° C.; TLC: Rf=0.37 (methanol/dichloromethane 10% v/v); 1H-NMR (DMSO-d6, 400 MHZ): 52.19 (3H, s, CH3), 2.36 (2H, m, CH2-CH2-CH2), 4.31 (2H, m, SO—CH2), 4.52 (1H, d, CO—CH2-CH2), 4.70 (2H, m, CH2-CH2-O), 4.91 (3H, m, CH2-CF3, CO—CH2-CH2), 6.30 (1H, d, J=9.6, Ar—H), 6.92 (1H, dd, J=8.6, 3.6, Ar—H), 6.98 (1H, s, Ar—H), 7.06 (1H, d, J=5.6, Ar—H), 7.50 (2H, m, Ar—H), 7.61 (1H, d, J=8.8, pyridine-CH), 7.86 (1H, d, J=7.6, Ar—H), 7.99 (1H, d, J=9.2, Ar—H), 8.06 (1H, d, J=8.0, Ar—H), 8.31 (1H, d, J=5.2, pyridine-CH); 13C-NMR (DMSO-d6, 100 MHZ): 5 11.4, 28.4, 60.4, 65.4, 65.8 (J2C—F=9), 66.8, 102.0, 107.7, 113.2, 113.3, 113.4, 115.7, 121.4, 122.9, 124.7 (J1C—F=276), 125.8, 127.0, 130.3, 134.4, 143.0, 145.1, 148.6, 150.0, 152.5, 156.2, 159.0, 161.1, 162.0, 162.4; 19F-NMR (DMSO-d6, 376 MHZ): 5-72.7 (3F, s); MS (ESI positive) m/z=616.1 [M+H]+. Comprehensive enzyme inhibition studies have been performed on human carbonic anhydrases. All hybrid compounds and individual CAI moieties with OH/SH group were tested for inhibition of off-target isoforms CA I and II and CAI IV, IX and XII by a Stopped-Flow kinetic assay. The inhibition profiles for the A1-A4 hybrid derivative set composed of the PPI lansoprazole linked via NH imidazole and carbamate linker to hydroxyalkyloxy coumarins as CAI are shown in Table 1.

TABLE 1

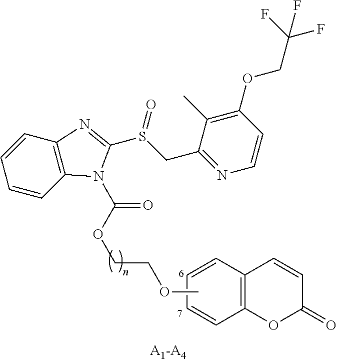

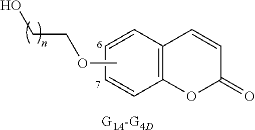

| | | | Ki (nM) | | | | |
|---|---|---|---|---|---|---|---|
| Cmpd | n | 6/7 | CA I | CA II | CA IV | CA IX | CA XII |
| A₁ | 1 | 6 | >10000 | >10000 | 14.4 | 79.2 | 53.0 |
| A₂ | 1 | 7 | >10000 | >10000 | 10.3 | 97.2 | 60.3 |
| A₃ | 2 | 6 | >10000 | >10000 | 6.4 | 41.6 | 8.9 |
| A₄ | 2 | 7 | >10000 | >10000 | 7.7 | 47.0 | 20.6 |
| G_{1A} | 1 | 6 | >10000 | >10000 | 6.8 | 305.6 | 250.9 |
| G_{1B} | 1 | 7 | >10000 | >10000 | 8.4 | 334.4 | 236.1 |
| G_{1C} | 2 | 6 | >10000 | >10000 | 7.2 | 218.0 | 91.4 |
| G_{1D} | 2\| | 7 | >10000 | >10000 | 7.6 | 243.3 | 160.7 |
| AAZ | — | — | 250 | 12 | 74 | 25 | 5.7 |

*Average of 3 experiments using the Stopped-Flow technique (errors of ± 5-10% of the indicated value)

Table 1 shows enzyme inhibition profiles against CAI I, II, IV, IX and XII of the hybrid derivative set A1-A4 and its CAI counterparts of the type G1 A-G1 D. In addition, cell growth inhibition studies were also performed. In vitro tests were performed on three different histotypes of human tumor cell lines: melanoma (me30966), glioblastoma (11373) and prostate cancer (LNCaP). A control experiment with isolated peripheral blood mononuclear cells was also included. The tumor lines were cultured at different pH conditions, both in buffered medium at pH 7.4 and in medium with acidic pH (6.5). Before being maintained in acidic pH medium, the tumor cells were cultured in unbuffered medium conditions so that they spontaneously acidified their culture medium as previously described (Logozzi et al 2018). The tumor cells in both conditions were treated both with the hybrid molecule SB3-105 (1-10-25 pM) and with the single inhibitors contained in the hybrid compound such as lansoprazole (10-25-50 pM) and the coumarin derivative SB3-107 (1-10-25 pM). All experiments were performed in triplicate. The effect of the individual compounds was evaluated 24 and 48 hours after treatment by analyzing cell mortality with the FACSalibur instrument, after incubation with the 0.04% Trypan Blue dye.

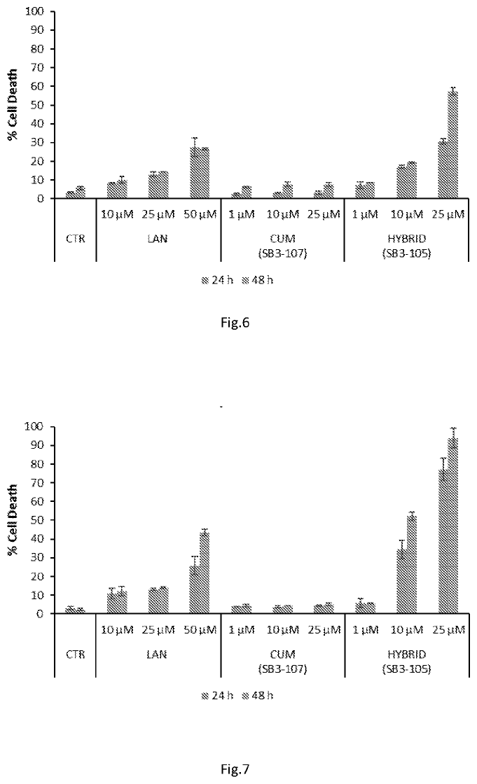

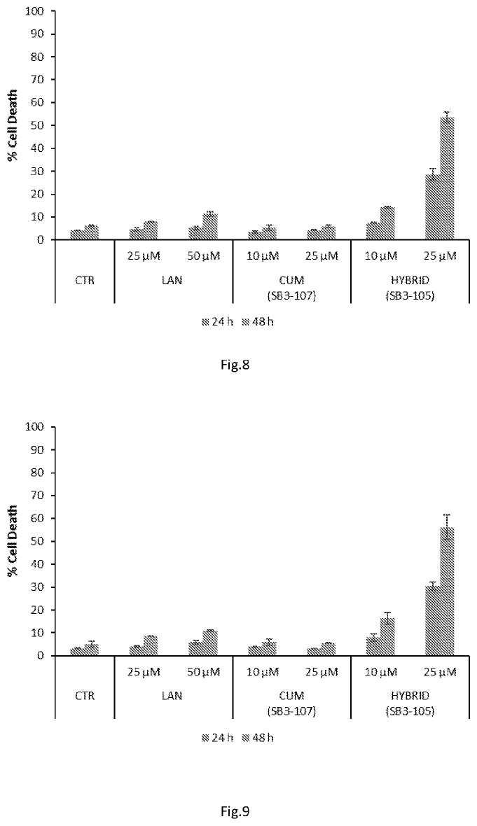

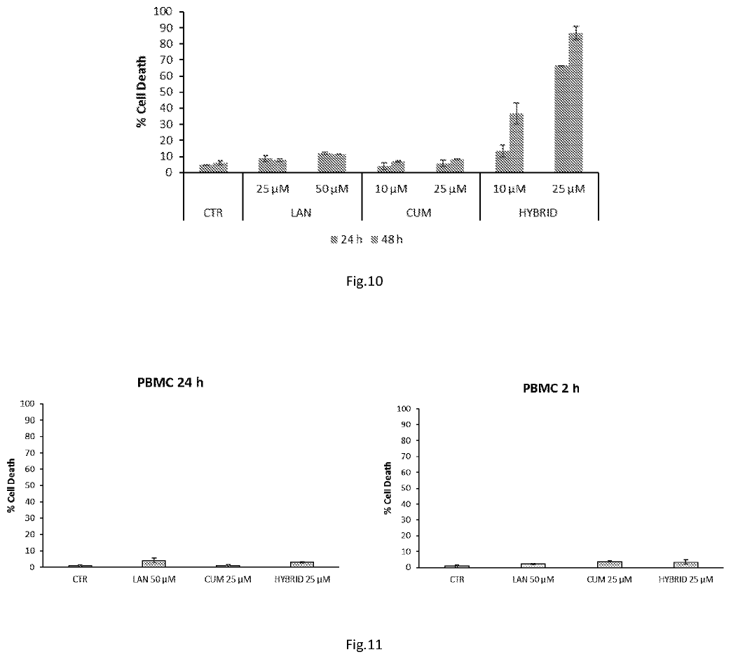

The results of the analyzes were reported in FIG. 6 and FIG. 7 (melanoma). The hybrid molecule showed high mortality in both culture conditions at pH 7.4 and pH 6.5. Furthermore, the hybrid molecule induces a much more potent cytotoxic effect at lower pH (6.5). The same experiments were performed with shorter treatment times, however showing detectable but much lower effects in both pH conditions (results not shown). Comparable results were obtained with glioblastoma cells, the results of the analyzes are reported in FIG. 8 cell mortality at pH 7.4 and FIG. 9 cell mortality at pH 6.5. Comparable results were obtained with prostate cancer cells, FIG. 10, cell mortality at pH 6.5. Further tests were conducted on peripheral blood mononuclear cells (PBMC). The results were obtained with peripheral blood mononuclear cells cultured at pH 7.4, FIG. 11. Even at the highest concentrations, the individual compounds of the hybrid molecule and the hybrid molecule itself show no cytotoxic effect on normal control cells (PBMC).

Advantageously, the new compositions according to the invention are used to suppress diseases characterized by acidosis such as, but not limited to, hypoxic and/or metastatic tumours; gastrointestinal disorders; inflammation; arthritis; pathogenic infections; to decrease or reduce gastrointestinal toxicity associated with the use of nonsteroidal anti-inflammatory compounds in a mammal.

Advantageously, the use of the new compositions is used for the treatment of diseases characterized by acidosis, based on the activation of hybrid prodrugs in the target tissue.

Advantageously, the use of the new compositions is used to reduce the acidity in the target tissue by means of a multi-target strategy consisting in the inhibition of the proton pumps concomitant with the inhibition of the carbonic anhydrases.

Advantageously, the mammal is a man.

While at least one exemplary embodiment has been presented in the summary and detailed description, it is to be understood that there are a large number of variations which are within the scope of the invention. Furthermore, it must be understood that the embodiment or embodiments presented are only examples which are not intended to limit in any way the scope of protection of the invention or its application or its configurations. Rather, the summary description and the detailed description provide the expert in the sector with a convenient guide for implementing at least one exemplary embodiment, it being clear that numerous variants can be made in the function and in the assembly of the elements described herein, without departing from the scope of protection of the invention as established by the attached claims and their technical-legal equivalents.

1. A pharmaceutical compositions (A, G1-G8) capable of inhibiting the ATP-dependent proton pump (V-ATPase) and carbonic a-anhydrases (CA IV, IX and XII), having the general formulae: 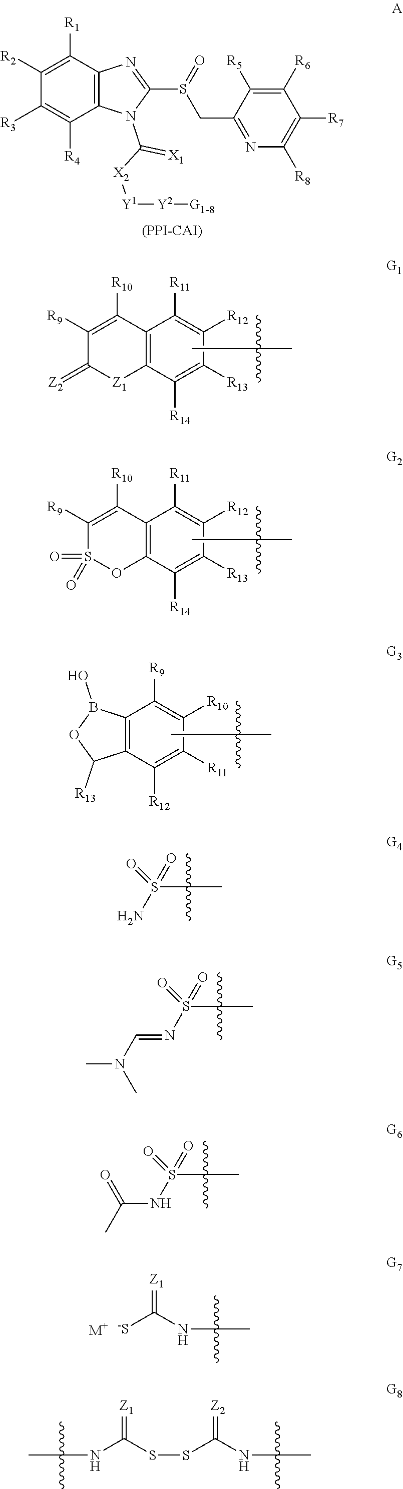
and a pharmaceutically acceptable excipient.

2. The compositions according to claim 1, wherein:
Gn=G1-G8;
X1, X2, Z1, Z2=O, S; —Y1, Y2=alkyl, alkenyl, alkynyl, cycloalkyl, heterocyclo, aryl, heteroaryl, ether, amine, ester, amide, anhydride, urea, thiourea, ketone, diazene, carbamate, thiocarbamate, sulfonamide, acylsulfonamide, acylurea; —R1, R2, R3, R4, R5, R6, R7, R8, R9, R10, R11, R12, R13, R14 are independently:
H, alkyl, alkenyl, alkynyl, cycloalkyl, heterocyclo, aryl, heteroaryl, halogen, hydroxy, ether, amine, ester, amide, anhydride, urea, thiourea, ketone, diazene, carbamate, thiocarbamate, sulfonamide, acylsulfonamide, acylurea or more of them;
M+=cation.

3. A use of the compositions according to claim 1, for treating acidosis of a patient.

4. The use of the compositions according to claim 1, for acidosis from hypoxic and/or metastatic tumors; gastrointestinal disorders; inflammation; arthritis; pathogenic infections; to decrease or reduce gastrointestinal toxicity associated with the use of non-steroidal anti-inflammatory compounds of a patient.

5. The use of the compositions according to claim 1, characterized by activating hybrid pro-drugs in the target tissue of a patient.

6. The use of the compositions according to claim 1, to reduce acidity in the target tissue of a patient by a multi-target strategy comprising inhibition of proton pumps concomitant with inhibition of carbonic anhydrases (CA).

7. The use of the compositions according to claim 1 for the treatment of acidosis characterized by a predetermined dosage of the pharmaceutical compositions (A, G1-G8).
